SEQUENCE LISTING (13 sequences)

SEQ ID NO: 1 - protein, 633 aa, synthetic construct
  source 1..633: note = SR21
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 2 - protein, 518 aa, synthetic construct
  source 1..518: note = SR22
MALPVTALLLPLALLLHAARPEVQVVESGGGTVQAGGSLRLSCAASARTFANAHMAWFRQAPGKEREFLAAITWSGGITDYANSVKGRFTISRDNAENAMYLQMNSLKPEDTAIYICAVDTTSAYDQLWSRQSEYEYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 3 - protein, 518 aa, synthetic construct
  source 1..518: note = SR23
MALPVTALLLPLALLLHAARPEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 4 - protein, 775 aa, synthetic construct
  source 1..775: note = SR24
MALPVTALLLPLALLLHAARPEVQVVESGGGTVQAGGSLRLSCAASARTFANAHMAWFRQAPGKEREFLAAITWSGGITDYANSVKGRFTISRDNAENAMYLQMNSLKPEDTAIYICAVDTTSAYDQLWSRQSEYEYWGQGTQVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 5 - protein, 775 aa, synthetic construct
  source 1..775: note = SR25
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSEVQVVESGGGTVQAGGSLRLSCAASARTFANAHMAWFRQAPGKEREFLAAITWSGGITDYANSVKGRFTISRDNAENAMYLQMNSLKPEDTAIYICAVDTTSAYDQLWSRQSEYEYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 6 - protein, 775 aa, synthetic construct
  source 1..775: note = SR26
MALPVTALLLPLALLLHAARPEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 7 - protein, 775 aa, synthetic construct
  source 1..775: note = SR27
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 8 - protein, 389 aa, synthetic construct
  source 1..389: note = SR28
METDTLLLWVLLLWVPGSTGDSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 9 - protein, 518 aa, synthetic construct
  source 1..518: note = SR29
METDTLLLWVLLLWVPGSTGDSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 10 - protein, 518 aa, synthetic construct
  source 1..518: note = SR210
METDTLLLWVLLLWVPGSTGDQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNHHHHHH

SEQ ID NO: 11 - protein, 523 aa, synthetic construct
  source 1..523: note = SR211
METDTLLLWVLLLWVPGSTGDSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSEVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSSHHHHHH

SEQ ID NO: 12 - protein, 523 aa, synthetic construct
  source 1..523: note = SR212
METDTLLLWVLLLWVPGSTGDEVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNHHHHHH

SEQ ID NO: 13 - DNA, 2519 bp, synthetic construct
  source 1..2519: mol_type = other DNA; note = mRNA IVT plasmid backbone
ggtttagtgaaccgtcagatccgctagagatccgcggccgctaatacgactcactataaggacatttgcttctgacacaactgtgttcactagcaacctcaaacagacaccgctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaccggtctagcataaccccttggggcctctaaacgggtcttgaggggttttttgcccgggggatcccctagggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttaaacggaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaaatgcggcctcgagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

(19) United States
(12) Patent Application Publication
(10) Pub. No.: US 2026/0201059 A1
(43) Pub. Date: Jul. 16, 2026

(54) ARMED CAR-MACROPHAGE COMPOSITIONS AND METHODS FOR THERAPY OF GLIOBLASTOMA MULTIFORME

(71) Applicant: RenaBio Therapeutics, Inc., Natick, MA (US)

(72) Inventors: Shengjun Ren, Newton, MA (US); Yu Zhang, Newton, MA (US)

(21) Appl. No.: 19/448,692

(22) Filed: Jan. 14, 2026

Related U.S. Application Data
(60) Provisional application No. 63/745,179, filed on Jan. 14, 2025.

(51) Int. Cl. C07K 16/30; A61K 9/51; A61K 31/7088; A61K 38/00; A61K 47/12; A61P 35/00; C07K 16/22; C07K 16/28; C12N 5/0786; C12N 15/11

(52) U.S. Cl. CPC C07K 16/30; A61K 9/5123; A61K 31/7088; A61K 38/00; A61K 47/12; A61P 35/00; C07K 16/22; C07K 16/2809; C07K 16/2866; C12N 5/0645; C12N 15/11; C07K 2317/31

(57) ABSTRACT

A nanoparticle for treating glioblastoma multiforme (GBM) in which the nanoparticle includes a lipid phase and a core that contains one or more nucleic acids encoding a chimeric antigen receptor (CAR), a T-cell engager (TCE), and/or a therapeutic gene. The CAR and the TCE each binding specifically to a GBM tumor-associated antigen and the nanoparticle selectively delivers the one or more nucleic acids to M2 macrophages in vitro and in vivo. Also provided is a therapeutic composition that contains a plurality of the nanoparticle described above and a pharmaceutically acceptable excipient. Further provided are methods for converting GBM tumor-resident M2 macrophages into M1 macrophages, and for treating GBM.

### CROSS REFERENCE TO RELATED APPLICATIONS

This application claims the priority of U.S. Provisional Application Ser. No. 63/745,179, filed Jan. 14, 2025, the content of which is hereby incorporated by reference in its entirety.

### REFERENCE TO AN ELECTRONIC SEQUENCE LISTING

A computer readable file containing a sequence listing is being electronically co-filed herewith via Patent Center. The computer readable file, submitted under 37 CFR § 1.831 (e), will also serve as the copy required by 37 § CFR 1.831 (c). The file (filename “RENA-003-101X.XML”) was created on Jan. 3, 2026, and has a size of 24,216 bytes. The content of the computer readable file is hereby incorporated by reference in its entirety.

### BACKGROUND

Adoptive cell therapy, such as chimeric antigen receptor (CAR) T cell therapy, has demonstrated great therapeutic efficacy in hematological malignancies. However, it has major drawbacks in treating solid tumors, in part, due to tumor microenvironment (TME) immunosuppression and antigen escape.

CAR T cell therapy requires a complicated and lengthy ex vivo manufacturing process. The extremely high cost of the therapy limits its accessibility to patients.

There is a critical need to develop adoptive cell therapy alternatives to traditional CAR T cell therapy, particularly for treating cancers such as glioblastoma multiforme, in the form of an off-the-shelf therapeutic product that has a lower cost and increased functionality that overcomes the drawbacks mentioned above.

### SUMMARY

To meet the above challenges, armed CAR-macrophage cell therapy is disclosed whose CAR gene, together with armed therapeutic modality genes are transferred into macrophage using an in vitro or in vivo (in situ) targetable nanoparticle to treat glioblastoma multiforme (GBM).

To accomplish this therapy, a nanoparticle for treating GBM is disclosed. The nanoparticle includes a lipid phase and a core that contains one or more nucleic acids. The core contains one or more nucleic acids that encode a chimeric antigen receptor (CAR), a T-cell engager (TCE), and/or a therapeutic gene. The CAR and the TCE each bind specifically to a GBM tumor-associated antigen The nanoparticle selectively delivers the one or more nucleic acids to M2 macrophages in vitro and in vivo.

Also provided is a therapeutic composition that contains a plurality of the nanoparticle described above and a pharmaceutically acceptable excipient.

Further provided is a method for converting GBM tumor-resident M2 macrophages into M1 macrophages. The method includes the step of contacting the M2 macrophages with a plurality of the nanoparticle set forth, supra.

Moreover, disclosed is a method for treating GBM in a subject. The method comprising administering to the subject a composition comprising the nanoparticle mentioned above in which the one or more nucleic acids encode (i) a CAR that specifically binds to a GBM tumor associated antigen (TAA), (ii) a T-cell engager (TCE) specifically binds to another GBM TAA and binds to a T cell, and (iii) a therapeutic gene that is effective against GBM.

The details of one or more embodiments of the invention are set forth in the description and drawings below. Other features, objects, and advantages of the invention will be apparent from the description, the drawings, and the claims.

### BRIEF DESCRIPTION OF THE DRAWINGS

The foregoing and other objects, features and advantages will be apparent from the following description of particular embodiments of the disclosure, as illustrated in the accompanying drawings.

FIG. 1 shows bar graphs of relative light units (RLU) of NFAT-Jurkat T cells (NFAT; T cells expressing luciferase under the control of the NFAT promoter) co-cultured with U87 glioblastoma cells (U87) and assayed 10 h after treatment with supernatant from M2 macrophages (negative control; NC) or supernatant from M2 macrophages transfected with the indicated T cell engager (TCE) mRNA lipid nanoparticle (LNP) construct. Supernatant from transfected M2 was collected 1, 2, 4, 7, or 10 days after TCE mRNA LNP transfection. Background luciferase activity was measured from cultures of U87 cells and co-cultures of NFAT cells and U87 cells.

FIG. 2 shows bar graphs of RLU of NFAT cells co-cultured with U251 glioblastoma cells (U251) and assayed 10 h after treatment with supernatant from M2 macrophages (negative control; NC) or supernatant from M2 macrophages transfected with the indicated TCE mRNA LNP construct. Supernatant from transfected M2 was collected 1, 2, 4, 7, or 10 days after TCE mRNA LNP treatment. Background luciferase activity was measured from cultures of U251 cells and co-cultures of NFAT cells and U251 cells.

FIG. 3 shows bar graphs of luciferase activity (expressed as tumor burden relative to untreated cells) of U138 glioblastoma cells stably expressing GFP and luciferase (U138_GFP/Luci) co-cultured with T cells assayed 3 days after treatment with supernatant from M2 macrophages transfected with the indicated TCE mRNA LNP construct (TCE). Supernatant from transfected M2 was collected 1, 2, 4, 7, or 10 days after TCE mRNA LNP treatment. The effector (T cell) to target (U138_GFP/Luci) ratio was 1/2.

FIG. 4 shows bar graphs of luciferase activity from a study similar to that shown in FIG. 44 except that U251 glioblastoma cells stably expressing GFP and luciferase (U251_GFP/Luci) was tested instead of U138_GFP/Luci.

FIG. 5 shows bar graphs of luciferase activity (expressed as tumor burden relative to glioblastoma cells+M2 macrophage+T cells) of U251_GFP/Luci cells co-cultured with M2 macrophages and T cells, either untreated (U251+M2+T) or treated with the indicated TCE mRNA LNP with the indicated E/T ratios (T cells to U251_GFP/Luci cells). Luciferase activities were determined 6 days after treatment with the TCE mRNA LNP at 1 μg./ml. The M2/U251_GFP/Luci E/T ratio was 1/8.

FIG. 6 shows bar graphs of tumor burden (normalized to untreated glioblastoma cell/M2 macrophage coculture) of U87_GFP/Luci cocultured with M2 macrophages untreated (NC) and treated with 1 μg/ml of the indicated chimeric antigen receptor mRNA LNP (CAR mRNA LNP SR21-SR27). Luciferase activity was determined 4 days after treatment. The E/T (M2 to U87_GFP/Luci) was 1/8.

FIG. 7 shows fluorescence microscopy images (upper panels) and corresponding phase contrast images (lower panels) of U87_GFP/Luci cells cocultured with M2 macrophages treated with 1 μg/ml of CAR^{SR27} mRNA LNP (left panels) or untreated (right panels) The images (10× magnification) were obtained 4 days post-treatment. The E/T for M2 was 1/8.

FIG. 8 shows bar graphs of luciferase activity (expressed as tumor burden relative to U87_GFP/Luci+M2+/−T) of U87_GFP/Luci cells co-cultured with M2 with or without T cells untreated (NC), treated with 2 μg/ml CAR^{SR27}mRNA LNP (CAR_SR27), 2 μg/ml TCE^{SR212} mRNA LNP (TCE_SR212) or 2 μg/ml TCE^{SR212} CAR^{SR27} mRNA LNP (TCE_SR212-CAR_SR27). Luciferase activity was measured 3 days after treatment. The E/T for M2 was 1/64 and for T cells 1/4.

FIG. 9 shows fluorescence microscopy images (upper panels) and corresponding phase contrast images (lower panels) of U87_GFP/Luci cells cocultured with M2 macrophages and T cells untreated (NC; right panels), treated with 2 μg/ml TCE^{SR212}-CAR^{SR27} mRNA LNP (left panels), and treated with 2 μg/ml TCE^{SR212} mRNA LNP (center panels). The images (10× magnification) were obtained 3 days post-treatment. The E/T for M2 was 1/64 and for T cells 1/4.

FIG. 10 shows bar graphs from a study similar to that shown in FIG. 8 except that U251_GFP/Luci was tested instead of U87_GFP/Luci. Details are found in the legend to that figure.

FIG. 11 shows fluorescence microscopy images and corresponding phase contrast images from a study similar to that described in FIG. 9 except that U251_GFP/Luci was tested instead of U87_GFP/Luci.

### DETAILED DESCRIPTION

As mentioned in the SUMMARY section, a nanoparticle for treating GBM is disclosed. The nanoparticle includes a lipid phase and a core that contains one or more nucleic acids.

The lipid phase includes an ionizable lipid, cholesterol, a phospholipid and a polyethylene glycol-conjugated lipid (pegylated lipid).

Ionizable lipids refer to a lipid or lipid-like material capable of being positively charged and able to electrostatically bind nucleic acids. As used herein, a “cationic lipid” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl, or more acyl chains, and the head group of the lipid typically carries the positive charge. Exemplary cationic lipids include one or more amine group(s) which bear the positive charge. Cationic lipids may encapsulate negatively charged RNA. In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “ionizable” lipids or lipid-like materials are comprised by the term “cationic lipid” or “cationic lipid-like material” unless contradicted by the circumstances. Examples of cationic lipids include, but are not limited to: ((4-hydroxybutyl) azanediyl)bis(hexane-6,1-diyl)bis(2-hexyldecanoate), 1,2-dioleoyl-3-trimethylammonium propane (DOTAP), N,N-dimethyl-2,3-dioleyloxypropylamine (DODMA), 1,2-di-O-octadecenyl-3-trimethylammonium propane (DOTMA), 3-(N—(N′,N′-dimethylamino-ethane) carbamoyl) cholesterol (DC-Chol), dimethyldioctadecylammonium (DDAB), 1,2-dioleoyl-3-dimethylammonium-propane (DODAP), 1,2-diacyloxy-3-dimethylammonium propanes, 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetra-decoxy) propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleoyloxypropyl-3-dimethyl-hydroxyethyl ammonium bromide (DORIE), 2,3-dioleoyloxy-N-[2 (spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethyl-aminopropane (DLenDMA), dioctadecylamidoglycyl spermine (DOGS), 3-dimethylamino-2-(cholest-5-en-3-beta-oxybutan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis,cis-9′,12′-octadecadienoxy)-propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethyl-propylamine (DLinDAP), 1,2-N,N′-dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethyl-aminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)-butanoate (DLin-MC3-DMA), N-(2-Hydroxyethyl)-N,N-dimethyl-2,3-bis(tetradecyloxy)-1-propanaminium bromide (DMRIE), (+)-N-(3-aminopropyl)-N,N-dimethyl-2,3-bis(cis-9-tetradecenyloxy)-1-propanaminium bromide (GAP-DMORIE), (+)-N-(3-aminopropyl)-N,N-dimethyl-2,3-bis(dodecyloxy)-1-propanaminium bromide (GAP-DLRIE), (+)-N-(3-amino-propyl)-N,N-dimethyl-2,3-bis(tetradecyloxy)-1-propanaminium bromide (GAP-DMRIE), N-(2-aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy) propan-1-aminium (DOBAQ), 2-({8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di [oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (di-methylamino)ethyl)thio)-carbonyl) azanediyl)dioctanoate (ATX), N,N-dimethyl-2,3-bis-(dodecyloxy) propan-1-amine (DLDMA), N,N-dimethyl-2,3-bis(tetradecyloxy) propan-1-amine (DMDMA), di((Z)-non-2-en-1-yl)-9-((4-(dimethylaminobutanoyl)oxy) heptadecanedioate (L319), N-dodecyl-3-((2-dodecylcarbamoylethyl)-{2-[(2-dodecylcarbamoylethyl)-2-{(2-dodecyl-carbamoylethyl)-[2-(2-dodecylcarbamoyl-ethylamino)-ethyl]-amino}ethylamino) propionamide (lipidoid 98N12-5), 1-[2-[bis(2-hydroxydodecyl)-amino]ethyl-[2-[4-[2-[bis(2 hydroxydodecyl)-amino]ethyl]piperazin-1-yl]ethyl]-amino]dodecan-2-ol (lipidoid 02-200); C12-200; or heptadecan-9-yl 8-((2-hydroxyethyl) (6-oxo-6-(undecyloxy) hexyl)amino) octanoate (SM-102). In some aspects, 1, 2, 3, 4, 5, or more of the foregoing cationic lipids may be excluded from the LNPs of the present disclosure.

Preferably, the ionizable lipid is (6Z,9Z,28Z,31Z)-heptatriaconta-6,9,28,31-tetraen-19-yl 4-(dimethylamino)butanoate (D-Lin-MC3-DMA), heptadecan-9-yl 8-((2-hydroxyethyl) (6-oxo-6-(undecyloxy) hexyl)amino) octanoate (SM102), 9Z,12Z-octadecadienoic acid, 3-[4,4-bis(octyloxy)-1-oxobutoxy]-2-[[[3-(diethylamino) propoxy]carbonyl]oxy]methyl]propyl ester (LP01), 6-((2-hexyldecanoyl)oxy)-N-(6-((2-hexyldecanoyl)oxy) hexyl)-N-(4-hydroxybutyl) hexan-1-aminium (ALC-0315), and analogs thereof. Analogs of the ionizable lipids include modifications of one or more of the head, chemical linkage, bridge chain, and tail portion.

The chemical nature changes of the ionizable lipid's head amine group, e.g., substituting different amine groups such as tertiary amines, secondary amines, or cyclic amines, can affect its pKa value, which impacts how well the head interacts with nucleic acids inside an acidic endosome.

The ionizable lipid's chemical linkage (bridge chain) connects the head group to the tail. Its alteration, e.g., introducing ester, amide, ether, or disulfide linkages, can influence the degradation rate and stability of the ionizable lipid. This alteration can also affect endosomal escape.

The modification of the ionizable lipid tail includes using different fatty acid chains with variations of chain lengths, unsaturation degree, and/or branching patterns. It can impact the ionizable lipid overall hydrophobicity and membrane-disrupting capabilities.

The ionizable lipid typically constitutes by mole percentage 5-95% (e.g., 37% to 47%, 10-90%, 15-85%, 20-80%, 25-70%, 30-60%, 35-50%, 38%, 39%, 40%, 41%, 42%, 43%, 44%, 45%, and 46%). Further a molar ratio of the ionizable lipid to the one or more nucleic acids is preferably in the range of 2:1 to 6:1 (e.g., 2.5:1 to 5.9:1, 3:1 to 5.8:1, and 3.3:1 to 5.6:1).

The nanoparticle further contains cholesterol or cholesterol derivative. Examples of cholesterol derivatives include, but are not limited to, cholestanol, 5,6-epoxy cholestanol, cholestanone, cholestenone, coprostanol, cholesteryl-2′-hydroxyethyl ether, cholesteryl-4′-hydroxybutyl ether, 24-ethyl cholesterol, 24-methyl cholesterol, cholenic acid, 3-hydroxy-5-cholestenoic acid, cholesteryl palmitate, cholesteryl arachidonate, cholesteryl arachidate, cholesteryl myristate, cholesteryl palmitoleate, cholesteryl lignocerate, cholesteryl oleate, cholesteryl stearate, cholesteryl erucate, cholesterol α-linolenate, cholesteryl linoleate, cholesteryl homo-γ-linolenate, 4-hydroxy cholesterol, 6-hydroxy cholesterol, 7-hydroxy cholesterol, 19-hydroxy cholesterol, 20-hydroxy cholesterol, 22-hydroxy cholesterol, 24-hydroxy cholesterol, 25-hydroxy cholesterol, 27-hydroxy cholesterol, 27-alkyne cholesterol, 7-keto cholesterol, 7-dehydro cholesterol, 8-dehydro cholesterol, 24-dehydro cholesterol, 5α-hydroxy-6-keto cholesterol, 20,22-dihydroxy cholesterol, 7,25-dihydroxy cholesterol, 7,27-dihydroxy cholesterol, 7-keto-25-hydroxy cholesterol, fucosterol, phytosterol, cholesteryl 11,14-eicosadienoate, dimethyl hydroxyethyl aminopropane carbamoyl cholesterol iodide and mixtures thereof. The cholesterol derivative may comprise a sugar moiety and/or amino acids. In an embodiment the amino acids are selected from serine, threonine, lysine, histidine, arginine or their derivatives.

The nanoparticle includes the cholesterol or cholesterol derivative in an amount typically ranging by mole percentage 1% to 90% (e.g., 42% to 50%, 5% to 85%, 10% to 80%, 15% to 75%, 20% to 70%, 25% to 65%, 30% to 60%, 35% to 55%, 43%, 44%, 45%, 46%, 47%, 48%, and 49%).

Exemplary phospholipids include phosphatidylcholines, e.g., diacylphosphatidylcholines, such as distearoylphosphatidylcholine (DSPC), dioleoylphosphatidylcholine (DOPC), dimyri-stoylphosphatidylcholine (DMPC), dipentadecanoylphosphatidylcholine, dilauroylphospha-tidylcholine, dipalmitoylphosphatidylcholine (DPPC), dioleoylphosphatidylglycerol (DOPG), dipalmitoylphosphatidylglycerol (DPPG), diarachidoylphosphatidylcholine (DAPC), dibehenoyl-phosphatidylcholine (DBPC), ditricosanoylphosphatidylcholine (DTPC), dilignoceroyl-phatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoylphosphatidylethanolamine (DOPE), 1,2-diundecanoyl-sn-glycero-phosphocholine (DUPC), palmitoyloleoylphosphatidylcholine (POPC), palmitoyloleoylphosphatidylethanolamine (POPE) and dioleoylphosphatidylethanolamine 4-(N-maleimidomethyl)-cyclohexane-1-carboxylate (DOPE-mal), dipalmitoyl phosphatidyl ethanolamine (DPPE), dimyristoylphospho-ethanolamine (DMPE), dilauroyl-phosphatidylethanolamine (DLPE), distearoylphosphatidyl-ethanolamine (DSPE), 1-phytanoyl-phosphatidylethanolamine (DpyPE), 16-O-monomethyl PE, 16-O-dimethyl PE, 18-1-trans PE, 1-stearoyl-2-oleoylphosphatidyethanolamine (SOPE), 1,2-dielaidoyl-sn-glycero-3-phosphoethanolamine (transDOPE), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphor-ethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, 1, 2, 3, 4, 5, or more of the foregoing neutral lipids may be excluded from the LNPs of the present disclosure. Without being bound by any theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from or from about 2:1 to about 8:1, or from or from about 10:0 to about 1:9, about 4:1 to about 1:2, or about 3:1 to about 1:1. In another aspect, the molar ratio of the cationic lipid to cholesterol ranges from or from about 2:1 to 1:1. In a further aspect, the molar ratio of the cationic lipid to the pegylated lipid ranges from or from about 100:1 to about 10:1 or from about 100:1 to about 25.1. In some aspects, the non-cationic lipid, e.g., neutral lipid (e.g., one or more phospholipids and/or cholesterol), may comprise from or from about 0 mol % to about 90 mol %, from or from about 0 mol % to about 80 mol %, from or from about 0 mol % to about 70 mol %, from or from about 0 mol % to about 60 mol %, or from or from about 0 mol % to about 50 mol %, of the total lipid present in the particle. In some aspects, the non-cationic lipid, e.g., neutral lipid (e.g., one or more phospholipids and/or cholesterol), may or may not be at least, at most, exactly, or between (inclusive or exclusive) of 0 mol %, 10 mol %, 20 mol %, 30 mol %, 40 mol %, 50 mol %, 60 mol %, 70 mol %, 80 mol %, or 90 mol % of the total lipid present in the particle. In some aspects, the phospholipid is in the range, by molar percentage, of 1% to 30% (e.g., 7% to 12%, 2% to 25%, 3% to 20%, 4% to 18%, 5% to 15%, 8%, 9%, 10%, and 11%).

Preferred phospholipids include dilinoleoylphosphatidylcholine (DLPC), dipalmitoylphosphatidylcholine (DPPC), distearoylphosphatidic acid (DSPA), dipalmitoylphosphatidic acid (DPPA), distearoylphosphatidylcholine (DSPC), dimyristoylphosphatidylethanolamine (DMPE), diheptanoylphosphatidylcholine (DHPC), dimyristoylphosphatidylcholine (DMPC), stearoylpalmitoyl-phosphatidylcholine (SPPC), and diarachidoylphosphatidylcholine (DAPC).

Further, the pegylated lipids can be polyethylene glycol (PEG)-lipid conjugates, such as PEG coupled to lipids (for example, DMG-PEG 2000), PEG coupled to phospholipids (for example, phosphatidylethanolamine (PEG-PE)), PEG conjugated to cholesterol or a derivative thereof, and mixtures thereof. In certain instances, PEG may be optionally substituted by alkyl, alkoxy, acyl, or aryl group.

Useful PEGs in the PEG-lipid conjugates are classified by their molecular weights (e.g., number average molecular weights); for example, PEG 2000 has an average molecular weight of about 2,000 Daltons, and PEG 5000 has an average molecular weight of about 5,000 Daltons. PEGs are commercially available from suppliers such as Avanti Polar Lipids. The PEG moiety of the PEG-lipid conjugates described herein may comprise a number-average molecular weight ranging from 550 Daltons to 20,000 Daltons (e.g., 600 Daltons to 15,000 Daltons, 800 Daltons to 10,000 Daltons, 900 Daltons to 8,000 Daltons, 1,000 Daltons to 6,000 Daltons, 1,200 Daltons to 5,000 Daltons, 1,300 Daltons to 4,000 Daltons, 1,400 Daltons to 3,500 Daltons, 1,500 Daltons to 3,000 Daltons, 1,600 Daltons, 1,800 Daltons, 1,900 Daltons, 2,000 Daltons, 2,100 Daltons, 2,200 Daltons, and 2,500 Daltons).

Suitable lipids for conjugating with PEG include phosphatidylethanolamines, diacylglycerols, phosphatidic acids, ceramides, dialkylamines, dialkylglycerols, 1,2-diacyloxypropan-3-amines, and mixtures thereof. These lipids have a variety of acyl chain groups of varying chain lengths and degrees of saturation. They are commercially available or can be isolated or synthesized using conventional techniques. The lipids can comprise saturated or unsaturated fatty acids with carbon chain lengths in the range of C10 to C20. They can contain mono- or polyunsaturated fatty acids and mixtures of saturated and unsaturated fatty acids. The lipids contemplated include, but are not limited to, dimyristoylglycerol (DMG), dimyristoylphosphatidylethanolamine (DMPE), dipalmitoyl-phosphatidyl ethanol amine (DPPE), dioleoylphosphatidylethanolamine (DOPE), and distearoylphosphatidylethanolamine (DSPE).

Suitable pegylated lipids are known in the art, e.g., WO2025238563A1. Examples include distearoylphosphatidylethanolamine-polyethylene glycol 2000 (DSPE-PEG 2000), dimyristoyl-PEG 2000 (DMG-PEG 2000), DSPE-PEG 2000-Mannose, DMG-PEG 2000-Mannose, PEG-c-DOMG, PEG-DLPE, PEG-DMPE, PEG-DPPC, PEG-ceramide conjugates (e.g., PEG-CerCI4 or PEG-CerC20), and PEG-c-DOMG. The pegylated lipid constitutes by molar percentage from 0.1% to 30% (e.g., 1.5% to 5.5%, 0.2% to 25%, 0.3% to 20%, 0.4% to 15%, 0.5% to 10%, 0.8% to 8%, 1% to 7%, 2%, 2.5%, 3%, 3.5%, 4%, 4.5%, and 5%).

Preferred pegylated lipids include distearoylphosphatidylethanolamine-polyethylene glycol 2000 (DSPE-PEG 2000), dimyristoyl-PEG 2000 (DMG-PEG 2000), DSPE-PEG 2000-Mannose, and DMG-PEG 2000-Mannose.

In certain embodiments, the lipid phase includes at least two pegylated lipids selected from DSPE-PEG 2000, DMG-PEG-2000, DSPE-PEG 2000-Mannose, and DMG-PEG-2000-Mannose.

The mole percentage of the ionizable lipid, the cholesterol, the phospholipid, and the pegylated lipid in the nanoparticle is 37% to 47%, 42% to 50%, 7% to 12%, and 1.5% to 5.5%, respectively.

As also mentioned above, the nanoparticle includes a core containing one or more nucleic acid. The core can include a buffer solution. The buffer can be at a pH of 4.5 to 6.5 at a concentration of 25 mM to 300 mM. Exemplary buffers include sodium citrate buffer and sodium acetate buffer.

The nanoparticle has a molar ratio of the ionizable lipid to the one or more nucleic acids ranging from 2:1 to 6:1, preferably 3:1 to 6:1, more preferably 3.3:1 to 5.6:1.

The one or more nucleic acid in the nanoparticle of the invention can encode a chimeric antigen receptor (CAR), a T-cell engager (TCE), and/or a therapeutic gene. In a particular embodiment, the nucleic acid is an mRNA molecule encoding the CAR TCE, or therapeutic gene. In a specific embodiment, the CAR, TCE, and therapeutic gene are encoded by separate mRNA molecules. Alternatively, a single mRNA molecule can encode the CAR, TCE, and therapeutic gene. In a preferred embodiment, the nanoparticle includes two individual mRNA molecules, one encoding the CAR and the other encoding the TCE.

Not to be bound by theory, the combination of the CAR and the TCE will expand the number of tumor-associated antigen (TAA) targets to overcome solid tumor associated genetic heterogeneity and therapy-induced targets loss. In addition, the activation and mobilization of the bystander T cells in the TME by the TCE will cross talk with macrophage to initiate neoantigen cancer vaccine in the treated solid tumor TME. It will alleviate TME immunosuppression and result in long term therapeutic efficacy.

The CAR includes a binding domain that specifically recognizes a GBM TAA. The TAA can be, but is not limited to, HER2, GD2, IL13Rα2, EGFR, EGFRvIII, CD133, CD44, SOX2, IDH1, TERT, tenascin-C, NY-ESO-1, MAGE-A, and GAGE.

In some embodiments, the CAR also includes a CD8α signal peptide, a CD8α hinge, a CD28 transmembrane domain, a CD3ζ signaling domain, and/or a 4-1BB costimulatory domain, and/or a TRL4-TIR domain, or combinations of these domains.

The CAR can have (include or consist of) the amino acid sequence of any one of SEQ ID NOs 1 to 7 or amino acid sequences having 75% to 99% similarity (75%, 80%, 85%, 90%, 95%, 99%) to SEQ ID NOs 1 to 7. Nucleic acids encoding these CAR are also within the scope of the invention.

The TCE can include a binding domain that specifically binds to a GBM TAA and a binding domain that specifically binds to T cells via CD3 or CD137 (4-1BB). In certain embodiments, the TCE includes two GBM TAA binding domains and a T cell binding domain.

The GBM TAA binding domain of the TCE can be, but is not limited to, HER2, GD2, IL13Rα2, EGFR, EGFRVIII, IL13Rα2, CD133, CD44, PD-L1, SOX2, IDH1, TERT, tenascin-C, NY-ESO-1, MAGE-A, and GAGE.

The TCE can have (include or consist of) the amino acid sequence of any one of SEQ ID NOs 8 to 12 or amino acid sequences having 75% to 99% similarity (75%, 80%, 85%, 90%, 95%, 99%) to SEQ ID NOs 8 to 12. Nucleic acids encoding these TCE are also within the scope of the invention.

In a particular nanoparticle, the mRNAs encode (i) a TCE having a GBM TAA binding domain and (ii) a CAR having the same GBM TAA binding domain as the TCE. Alternatively, the GBM TAA binding domain of the TCE is distinct from the GBM TAA binding domain of the CAR. Further, in an embodiment where the TCE includes two GBM TAA binding domains, the two binding domains can bind to distinct GBM TAAs or to the same GBM TAA.

As mentioned above, the mRNA can also encode a therapeutic gene. The therapeutic gene provides additional anti-cancer efficacy to the therapy. The therapeutic gene can be, e.g., an anti-VEGF antibody, an anti-VEGFR antibody, IL-2, IL-2 muteins, TGFβ-trap, TGFβ receptor muteins, anti-PD-1 antibody, anti-PD-L1 antibody, and anti-CD47 antibody.

The following method can be used to prepare the nanoparticle of the invention. The lipid phase components are mixed together at room temperature in a solvent, e.g., absolute ethanol, in the molar percentages set forth, supra. Separately, the one or more nucleic acid is formulated, also at room temperature, in a solution containing a buffer, e.g., sodium acetate buffer, to stabilize the nucleic acid. The lipid phase and solution are then mixed using an automated mixing instrument in a 1:1 to 1:5 v/v ratio of lipid phase to solution with a flow rate of 2 to 100 mL per minute at room temperature.

Also within the scope of the invention is a therapeutic composition that contains a plurality of the nanoparticle described above, together with a pharmaceutically acceptable excipient, e.g., solvents, salts, and sugars/cryoprotectants.

Another method is provided for converting GBM tumor-resident M2 macrophages into M1 macrophages by contacting the M2 macrophages with a plurality of the above-described nanoparticle. The contacting step is carried out in vivo by delivering the composition into a GBM tumor that contains M2 macrophages. Delivering the composition into a GBM tumor can be accomplished by a route that includes, but is not limited to, intracerebroventricular injection, intra-tumor injection, and intraspinal injection.

As summarized above, also disclosed is a method for treating GBM in a subject by administering a nanoparticle of the invention to a subject suffering from GBM. The nanoparticle includes one or more nucleic acids that encode a CAR, a TCE, and, optionally, a therapeutic gene. The CAR, TCE, and therapeutic gene are those described above. An exemplary nanoparticle includes in its core an mRNA encoding a CAR that specifically binds to HER2, EGFR, or IL13Rα2, an mRNA encoding a TCE that specifically binds to a GBM TAA and to CD3, and an mRNA encoding an anti-VEGF antibody.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications, including patent documents, cited herein are incorporated by reference in their entirety.

### EXAMPLES

### Example 1: Identification of GBM Lead TCE mRNA LNP with High Potency at a Low E/T Ratio

In addition to TAM-induced immune suppression in GBM, genetic heterogeneity is another key challenge to develop effective therapy for GBM. Tumor-associated antigen (TAA) expression was determined by flow cytometry in several cultured GBM cell lines. The results are shown in Table 1 below.

TABLE 1
Expression of GBM TAA in GBM cell lines

GBM Tumor-Associated Antigen

| GBM cell line | EGFR (%) | HER2 (%) | IL13Rα2 (%) |
|---|---|---|---|
| U138 | 99.40 | 0 | 4.61 |
| U87 | 99.50 | 0.48 | 37.78 |
| U251 | 99.90 | 87.60 | 96.10 |

A series of anti-IL13Rα2 and/or EGFR TCEs were constructed based on the TAA expression data (TCE_SR28-212; see Table 2 below).

TCE mRNA were synthesized by in vitro transcription (IVT) after inserting the corresponding cDNAs into the mRNA IVT construct backbone (SEQ ID NO: 13; see Table 2).

TABLE 2
CAR and TCE specificities and corresponding SEQ ID NOs

| Construct | Specificities | SEQ ID NO |
|---|---|---|
| CARSR21 | anti-HER2 | SEQ ID NO: 1 |
| CARSR22 | anti-EGFR | SEQ ID NO: 2 |
| CARSR23 | anti-EGFR | SEQ ID NO: 3 |
| CARSR24 | anti-HER2/EGFR | SEQ ID NO: 4 |
| CARSR25 | anti-HER2/EGFR | SEQ ID NO: 5 |
| CARSR26 | anti-HER2/EGFR | SEQ ID NO: 6 |
| CARSR27 | anti-HER2/EGFR | SEQ ID NO: 7 |
| TCESR28 | anti-IL13Rα2/CD3 | SEQ ID NO: 8 |
| TCESR29 | anti-EGFR/IL13Rα2/CD3 | SEQ ID NO: 9 |
| TCESR210 | anti-EGFR/IL13Rα2/CD3 | SEQ ID NO: 10 |
| TCESR211 | anti-EGFR/IL13Rα2/CD3 | SEQ ID NO: 11 |
| TCESR212 | anti-EGFR/IL13Rα2/CD3 | SEQ ID NO: 12 |
| mRNA IVT Plasmid | N/A | SEQ ID NO: 13 |

IVT was performed with pseudo-uridine and a capping reagent. LNP lipid components are described above. Exemplary LNPs could selectively transfect M2 macrophages with high efficiency. See Table 3 below.

TABLE 3
LNP formulations

| | | Molar Ratio of | | |
|---|---|---|---|---|
| mRNA | | Ionizable Lipid | % of | Expression |
| LNP | | to Nucleic | Transfected | Efficacy |
| Type | Pegylated Lipid | Acids | Cells | (+) |
| LNP_A | DMG-PEG-2000- | 4.5:1 | 25 | + |
| | Mannose | | | |
| LNP_B | DMG-PEG-2000 | 4.5:1 | 95 | ++++ |
| LNP_C | DSPE-PEG 2000 | 4.5:1 | 11 | + |
| LNP_D | DSPE-PEG 2000- | 4.5:1 | 12 | + |
| | Mannose | | | |
| LNP_E | 75% of DMG- | 3.5:1 | 95 | +++ |
| | PEG-2000 | | | |
| | 25% of DSPE- | | | |
| | PEG 2000- | | | |
| | Mannose | | | |

The mRNA LNPs were produced with the iNanno L as directed by the manufacturer (MicroNano) using its single molecule detector (SMD) card at room temperature. The mRNA LNPs were stored in 10% (w/v) sucrose at −80° C. The LNP particle size, surface charge and concentration were analyzed using Zeta Sizer Red.

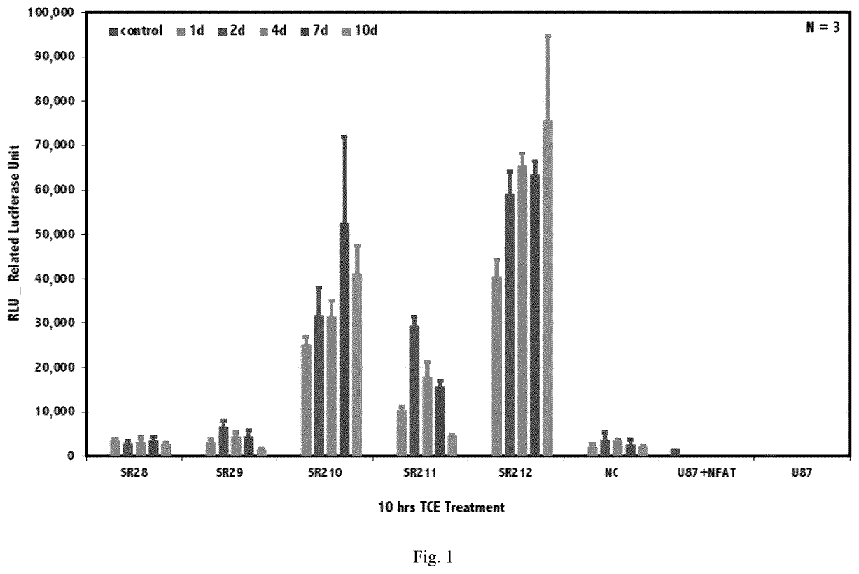

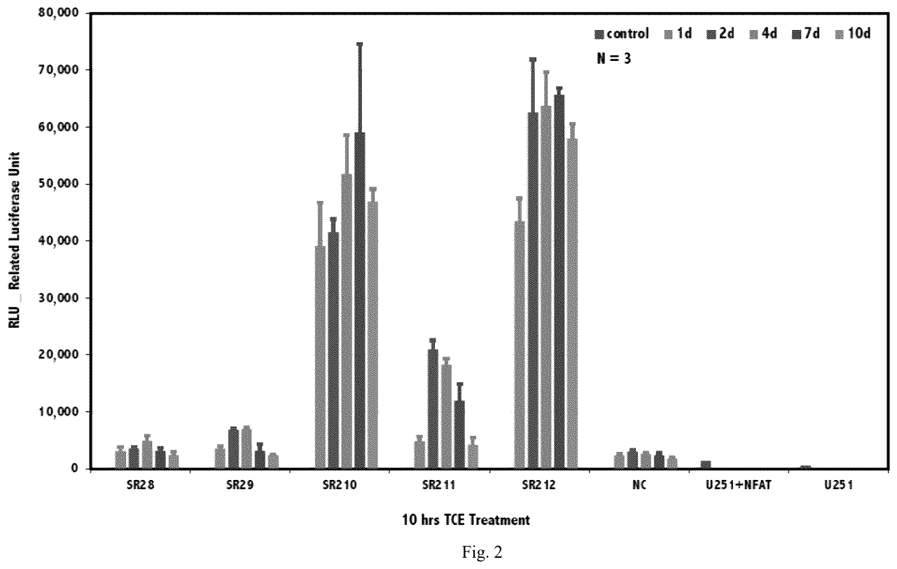

The TCE mRNA LNPs were first tested for their ability to activate T cells in the presence of GBM cells using a Jurkat NFAT-luciferase coculture system. GBM cancer cells at a concentration of 20,000 cells/well were plated in 96-well plates, and on the next day, Jurkat (NFAT-Luciferase) reporter cells as well as TCEs collected from mRNA LNP treated M2 macrophage supernatants were added at an E/T ratio (Jurkat/GBM) of 1/2 for 10, 12, or 24 hrs. Luciferase activity was then assessed using ONE-Step™ Luciferase assay system and luminescence was measured in a microplate reader. The results are shown in FIG. 1 for U87 cells and in FIG. 2 for U251 cells.

The secreted TCE from TCE^{SR212} mRNA LNP transfected M2 macrophages was the most efficient activator of T cells for both cell lines.

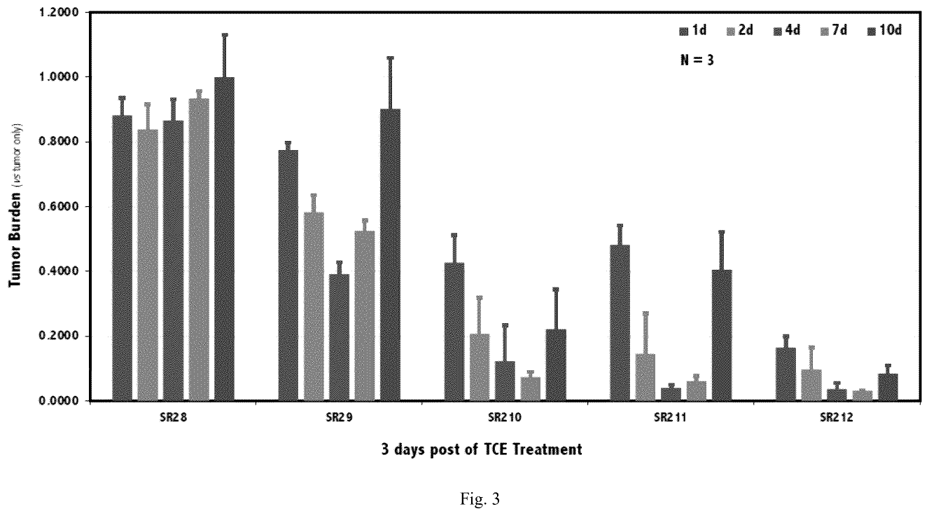

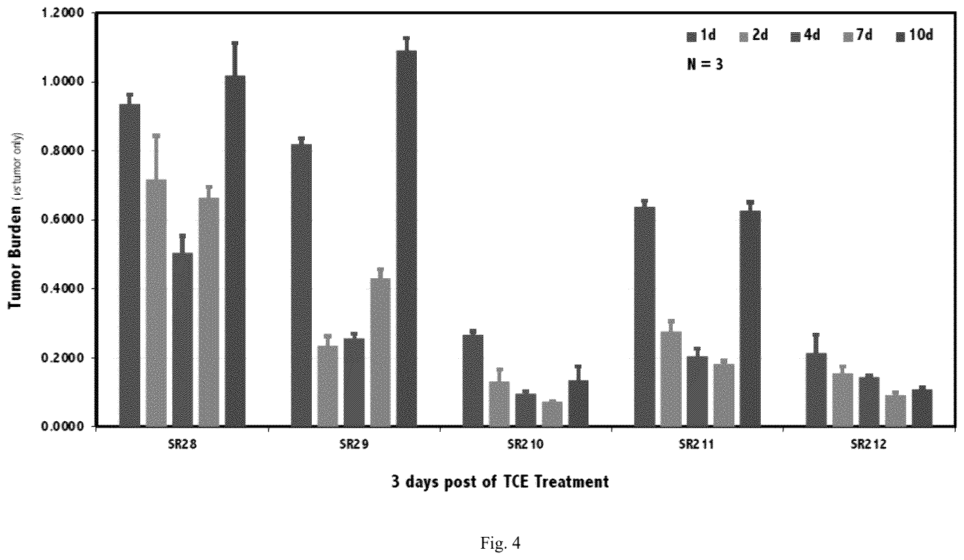

The ability of the secreted TCE to activate T cells to kill GBM cells was tested by treating cultured U138_GFP/Luci cells and U251_GFP/Luci cells with Pan-T cells and the secreted TCE. The results are shown in FIGS. 3 (U138) and 4 (U251). The results confirmed that TCE_SR212 was the most effective for killing GBM cells in the presence of T cells.

U251_GFP/Luci cells were cocultured with M2 macrophages and T cells and treated with the TCE mRNA LNP constructs described above. The results (see FIG. 5) showed that TCE_SR210 and TCE_SR212 were the most effective for killing the GBM cells.

### Example 2: Identification of GBM Lead CAR mRNA LNP with High Potency at a Low E/T Ratio

A series of anti-HER2 and/or EGFR CARs were constructed with differentiated intracellular stimulation/activation domains (constructs CAR_SR21-SR27; see Table 3 above). mRNA for each construct was prepared by in vitro transcription and encapsulated into LNP as described above to produce CAR mRNA LNPs.

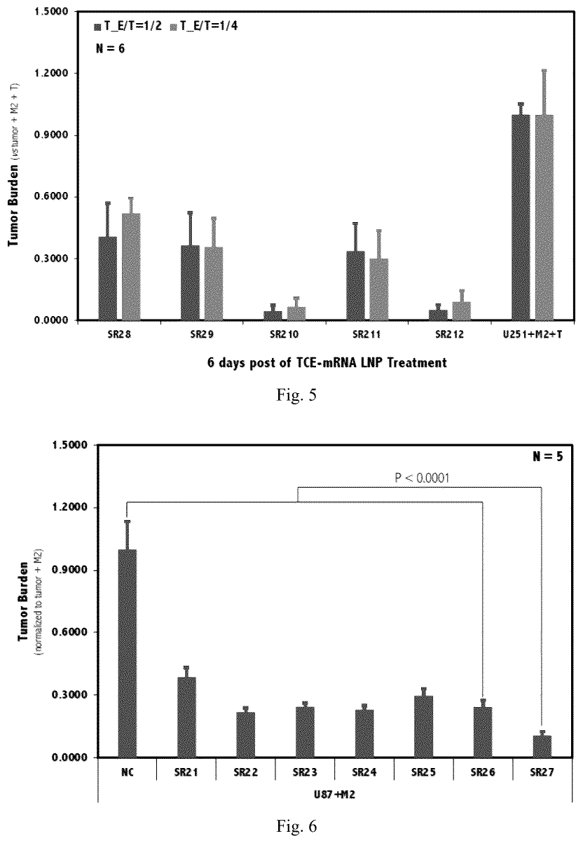

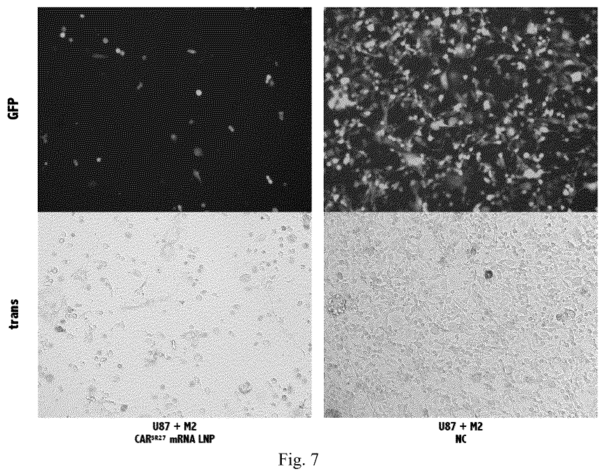

U251 GBM cells stably expressing both GFP and luciferase (U87_GFP/Luci) were co-cultured with M2 macrophages produced as described above at an effector (M2) to target (GBM) ratio of ⅛ and treated with the CAR mRNA LNPs. Luciferase activities in the cocultures were measured 3 days after CAR mRNA LNP treatment and compared to luciferase in cocultures not treated with the CAR mRNA LNPs. The results are shown in FIG. 6. CAR_SR27 was the most effective against GBM cells, killing nearly 80% of the U87_GFP/Luci cells.

The results were confirmed by direct imaging of GFP in the transfected U87 cells cocultured with M2 macrophages. See FIG. 7.

### Example 3: Identification of GBM Lead TCE_CAR mRNA LNP with High Potency at Very Low E/T Ratio

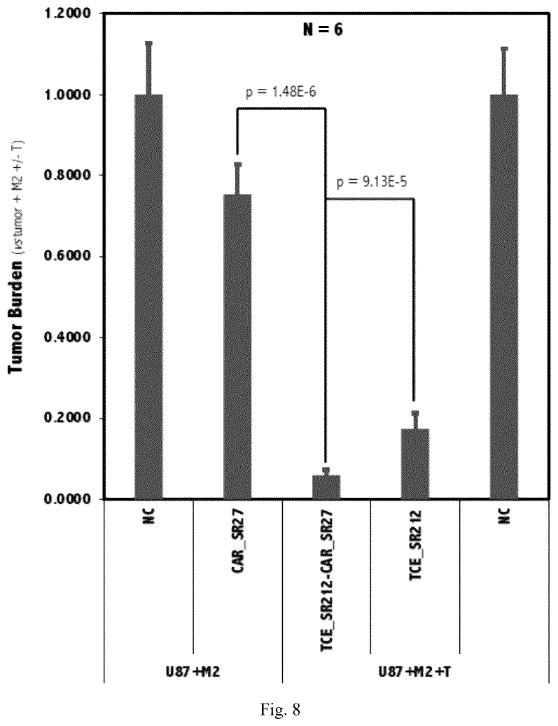

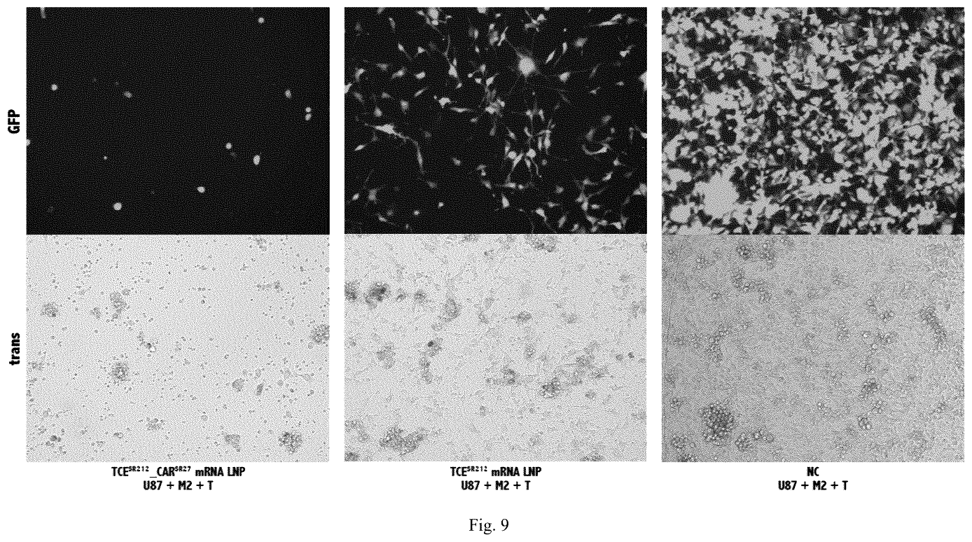

After identification of lead CAR and TCE for treating GBM, both CAR and TCE mRNAs were simultaneously encapsulated into M2 macrophage targeting LNP. The resulting TCE_CAR mRNA LNP (i.e., TCE^{SR212} CAR^{SR27} mRNA LNP) was tested for its ability to kill GBM cells U87_GFP/Luci and U251_GFP/Luci) cocultured with M2 macrophages and T cells. The results are shown in FIGS. 8 and 9.

Treatment of U87_GFP/Luci/M2/T cell cocultures with TCE^{SR212} CAR^{SR27} mRNA LNP resulted in significantly more killing of the GBM cells, as compared to TCE^{SR212} mRNA LNP, even though the TCE^{SR212} CAR^{SR27} mRNA LNP carried half the amount of RNA encoding TCE^{SR212}. See FIG. 8. Treatment with TCE^{SR212} CAR^{SR27} mRNA LNP was also more effective than CAR^{SR27} mRNA LNP treatment of GBM/M2 coculture. See FIG. 8. The effectiveness of TCE^{SR212} CAR^{SR27} mRNA LNP was even more remarkable given the E/T for M2 of 1/64 and the E/T for T cells of 1/4.

The effectiveness of the TCE_CAR mRNA LNP was confirmed by directly observing GFP expression in the U87_GFP/Luci cells cocultured with M2 and T cells and treated with TCE^{SR212} CAR^{SR27} mRNA LNP. See FIG. 9.

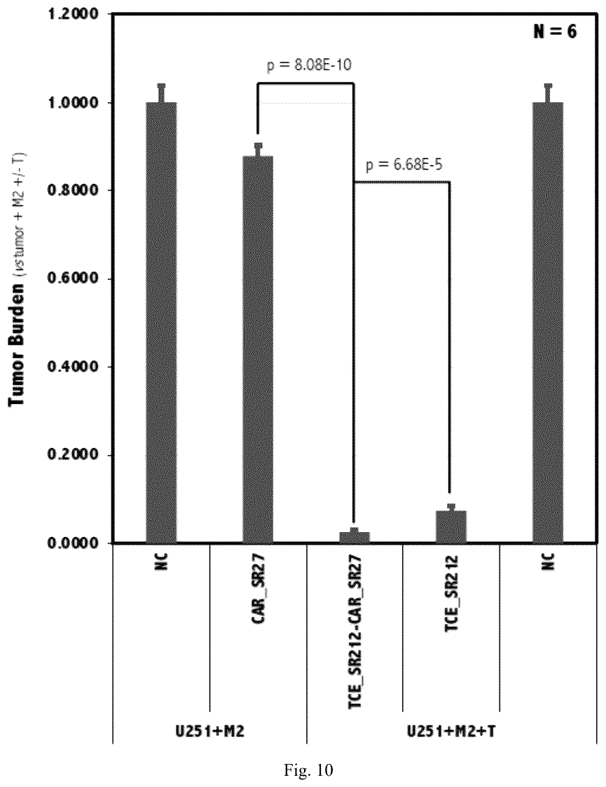

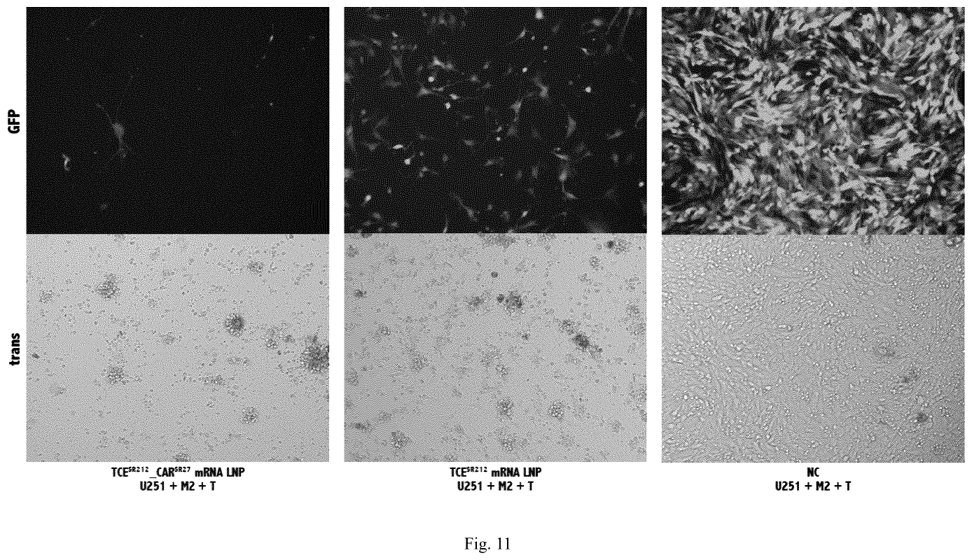

A similar study showed that the potency of the TCE^{SR212} CAR^{SR27} mRNA LNP for killing another GBM cell line, i.e., U251_GFP/Luci, cocultured with M2 and T cells (at the same E/T) was significantly better than the TCE mRNA LNP and the CAR mRNA LNP alone. See FIG. 10.

The effectiveness of the TCE_CAR mRNA LNP was confirmed by directly observing GFP expression in the U251_GFP/Luci cells cocultured with M2 and T cells and treated with TCE^{SR212} CAR^{SR27} mRNA LNP. See FIG. 11.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A nanoparticle for treating glioblastoma multiforme (GBM), the nanoparticle comprising a lipid phase and a core, the core comprising one or more nucleic acids that encode a chimeric antigen receptor (CAR), a T-cell engager (TCE), and, optionally, a therapeutic gene, wherein the CAR and the TCE each bind specifically to a GBM tumor-associated antigen and the nanoparticle selectively delivers the one or more nucleic acids to M2 macrophages in vitro or in vivo.

2. The nanoparticle of claim 1, wherein the lipid phase includes:
an ionizable lipid;
cholesterol or its derivative;
a phospholipid; and
a polyethylene glycol-conjugated lipid (pegylated lipid), and
the core further comprises a buffer solution.

3. The nanoparticle of claim 2, wherein a mole percentage of the ionizable lipid, the cholesterol or derivative, the phospholipid, and the pegylated lipid in the nanoparticle is 37% to 47%, 42% to 50%, 7% to 12%, and 1.5% to 5.5%, respectively, and a molar ratio of the ionizable lipid to the one or more nucleic acids is 2:1 to 6:1.

4. The nanoparticle of claim 2, wherein the ionizable lipid is selected from D-Lin-MC3-DMA, SM102, LP01, ALC-0315, and analogs thereof.

5. The nanoparticle of claim 2, wherein the phospholipid is selected from DLPC, DPPC, DSPA, DPPA, DSPC, DMPE, DHPC, DMPC, SPPC, and DAPC.

6. The nanoparticle of claim 5, wherein the pegylated lipid is selected from DSPE-PEG 2000, DMG-PEG-2000, DSPE-PEG 2000-Mannose, and DMG-PEG-2000-Mannose.

7. The nanoparticle of claim 6, wherein the lipid phase includes at least two pegylated lipids selected from DSPE-PEG 2000, DMG-PEG-2000, DSPE-PEG 2000-Mannose, and DMG-PEG-2000-Mannose.

8. The nanoparticle of claim 1, wherein the CAR specifically binds to one or two of HER2, EGFR, EGFRVIII, and IL13Rα2, the TCE binds specifically to a GBM TAA and to CD3, and the therapeutic gene, if present, is an anti-VEGF antibody.

9. The nanoparticle of claim 8, wherein the CAR has the amino acid sequence of any one of SEQ ID NOs 1 to 7.

10. The nanoparticle of claim 9, wherein the TCE has the amino acid sequence of any one of SEQ ID NOs 8 to 12.

11. The nanoparticle of claim 10, wherein the CAR has the amino acid sequence of SEQ ID NO: 7 and the TCE has the amino acid sequence of SEQ ID NO: 12.

12. A therapeutic composition for treating GBM, comprising a plurality of the nanoparticle of claim 1 and a pharmaceutically acceptable excipient.

13. A method for converting GBM tumor-resident M2 macrophages into M1 macrophages, the method comprising contacting the M2 macrophages with a plurality of the nanoparticle of claim 1.

14. A method for treating GBM in a subject, the method comprising administering to the subject a composition comprising the nanoparticle of claim 1, wherein the CAR specifically binds to one or two of HER2, EGFR, EGFRVIII, and IL13Rα2, the TCE specifically binds to EGFR, IL13Rα2, or both, and binds to a T cell, and the therapeutic gene, if present, is effective against GBM.

15. The method of claim 14, wherein the CAR has the amino acid sequence of any one of SEQ ID NOs 1 to 7.

16. The method of claim 15, wherein the TCE has the amino acid sequence of any one of SEQ ID NOs 8 to 12.

17. The method of claim 14, wherein the CAR has the amino acid sequence of SEQ ID NO: 7 and the TCE has the amino acid sequence of SEQ ID NO: 12.

18. The method of claim 14, wherein the lipid phase comprises a pegylated lipid selected from DSPE-PEG 2000, DMG-PEG-2000, DSPE-PEG 2000-Mannose, DMG-PEG-2000-Mannose, and mixtures thereof.

19. The method of claim 18, wherein the pegylated lipid is DMG-PEG-2000.

20. The method of claim 14, wherein the therapeutic gene increases the anti-cancer efficacy of the composition, as compared to the combined anti-cancer efficacy of the CAR and TCE.

21. A chimeric antigen receptor (CAR) for treating GBM, the CAR having the amino acid sequence selected from SEQ ID NOs 1 to 7.

22. An isolated nucleic acid encoding the CAR of claim 21.

23. A T Cell Engager (TCE) for treating GBM, the TCE having the amino acid sequence selected from SEQ ID NOs 8 to 12.

24. An isolated nucleic acid encoding the TCE of claim 23.
